(19) United States
(12) Patent Application Publication
(10) Pub. No.: US 2018/0044635 A1
(43) Pub. Date: Feb. 15, 2018

(54) SURFACE-MODIFIED MACROPHAGES FOR CELL-BASED DELIVERY

(71) Applicant: University of Massachusetts, Boston, MA (US)

(72) Inventor: Michelle Farkas, Amherst, MA (US)

(21) Appl. No.: 15/674,085

(22) Filed: Aug. 10, 2017

Related U.S. Application Data
(60) Provisional application No. 62/373,274, filed on Aug. 10, 2016.

(51) Int. Cl. C12N 5/0786; A61B 5/055; A61B 5/00; C12N 9/64

(52) U.S. Cl. CPC C12N 5/0645; C12N 9/6416; A61B 5/055; A61B 5/0073

(57) ABSTRACT

A population of isolated mammalian macrophages, the cell surface of which is chemically modified to comprise a molecule that comprises an imaging agent or a therapeutic agent or a molecule that binds to an imaging agent or a therapeutic agent, and methods of making and using the modified macrophages are provided.

### CROSS-REFERENCE TO RELATED APPLICATIONS

This application claims the benefit of the filing date of U.S. application Ser. No. 62/373,274, filed on Aug. 10, 2016, the disclosure of which is incorporated by reference herein.

### BACKGROUND

The processes of tumor invasion and establishment of distant metastases are the boundaries beyond which manageable cancers become untreatable: metastasis remains the principal cause of death for afflicted patients. Currently available means for cancer imaging and therapies are largely based upon the premise that tumor tissue will take up those agents more quickly than will normal tissue. This detection strategy has a strong propensity to yield both false negatives and positives, and is generally unable to reveal metastases or nascent disease over background signal. Similarly, where traditional, un-targeted therapeutics are concerned, the result may be broad toxicity to non-targeted organs. Medical professionals always have to weigh the costs versus benefits of treatment, hoping that the drugs kill the cancer before they kill the patient.

Macrophages are immune cells that play significant roles in cancer progression and metastasis, leading to the correlation of their presence with disease severity in many cancer types. Tumor-associated macrophages (TAMs) have been shown to generate factors that promote tumor angiogenesis, silence the immune response to tumors, and contribute to the epithelial to mesenchymal transition (or EMT, where epithelial cells undergo changes that result in an enhanced migratory capability, increased invasiveness, and elevated resistance to apoptosis ascribed to mesenchymal (stem-like) cell phenotypes) via remodeling of the tumor environment and association with tumor cells. They have also been implicated in the metastasis-enabling processes of intra- and extra-vasation of migratory tumor cells and can affect the efficacy of anti-cancer therapeutics. TAMs enhance tumor growth and invasion by generating angiogenic and stromal breakdown factors such as vascular endothelial growth factor (VEGF), epidermal growth factor (EGF), matrix metalloproteases (MMPs), and interleukin (IL)-17, and suppressing anti-tumoral immune responses via IL-10 and transforming growth factor (TGF)-β. TAMs are not only important in the initial stages of metastasis, but have been shown to contribute to the establishment and survival of metastases at sites away from the primary tumor. TAMs have been associated with a variety of tumor types, including breast, prostate, glioma, lymphoma, bladder, lung, cervical, and melanoma, and their increased presence has been correlated with malignancy and poor prognoses. In some breast cancers, macrophages have been reported to comprise up to 50% of the tumor cell mass.

Macrophages are plastic cells: their phenotype depends on their location and the physiological or pathological context present. There is a spectrum of macrophage activation, but they can be most broadly classified based on their polarization state as M1 or M2. M1 is the ‘classically-activated’ phenotype that is responsible for pro-inflammatory immune responses, including pathogen engulfment and breakdown, and immune cell (including other macrophage) stimulation. These macrophages typically promote anti-tumoral responses by activating the adaptive immune system, but are also capable of attacking cancer cells themselves. The M2 phenotype is involved in tissue repair/wound healing and resolution of inflammation. These cells suppress the immune response. TAMs, which assist in oncogenic progression, are considered ‘M2-like’. It has been shown that conditioned media from malignant epithelial cells can polarize macrophages to an M2-like phenotype, and that macrophages can also be “re-educated” to reverse their tumor promoting activity and produce an anti-tumor M1 phenotype

### SUMMARY

Macrophages localize to cancers and so can be used as imaging and/or therapeutic agents, e.g., by conjugating chemical entities to their surfaces. Both cancers and atherosclerotic plaques have been shown to produce two of the major chemo-attractants for macrophage cells. To take advantage of this active recruitment, macrophage-based agents were generated for the selective delivery of imaging and/or therapeutic entities via chemical modification of their cell surfaces. Such reagents may be employed for personalized imaging and/or treatment by isolating a patient's own macrophages, modifying and re-introducing them, using the power of the immune system to find and fight disease. Thus, surface modified macrophages provide for improved selectivity for imaging and/or drug agent delivery.

Thus, a cell-based platform is provided herein that can facilitate imaging and/or therapeutic agent delivery with high levels of specificity. In one embodiment, a population of isolated mammalian macrophages is chemically modified to comprise a molecule comprising an imaging agent or a therapeutic agent or a molecule that binds to (e.g., a chelator that binds to a metal) an imaging agent or therapeutic agent. In one embodiment, the molecule comprising the imaging agent or therapeutic agent comprises a linker which separates the imaging agent or therapeutic agent from the cell surface. In one embodiment, there is no linker that separates the molecule comprising the imaging agent or therapeutic agent from the cell surface. In one embodiment, the cells are modified with a molecule, e.g., a chelator, which itself binds to the imaging agent or therapeutic agent. In one embodiment, the cells are modified with a linker that interacts with the imaging agent or therapeutic agent via non-covalent interactions, e.g., electrostatic interactions. In one embodiment, the population comprises immortalized macrophages or macrophage-precursors. In one embodiment, the population comprises human macrophages. In one embodiment, the modification comprises a covalent bond between a native cell surface molecule and a molecule that comprises an imaging agent or binds to an imaging agent. In one embodiment, the imaging agent is for position emission tomography. In one embodiment, the imaging agent is for magnetic resonance imaging. In one embodiment, the modification comprises a linker. In one embodiment, the linker is a cleavable linker. In one embodiment, the linker comprises a hydrazone, Schiff base, N-hydroxysuccinamide (NETS), or thiomaleamic acid. In one embodiment, the linker comprises a peptide substrate for a protease, e.g., for a matrix metalloproteinase (MMP). In one embodiment, the substrate comprises Pro-Leu-Gly-Met-Trp-Ser-Arg or His-Val-Leu-Asn-Leu-Arg-Ser-Thr. In one embodiment, the peptide substrate is a MMP-2 or MMP-9 substrate. In one embodiment, the modification comprises an azide-functionalized sugar, e.g., N-azidoacetylmannosamine (ManNaz), N-azidoacetylgalactosamine, azidoacetylglucoseamine (GluNaz) or 6-azidofucose. In one embodiment, the molecule comprises phosphine. In one embodiment, the therapeutic agent comprises a chemotherapeutic. In one embodiment, each macrophage is modified with at least 1, 10, 100, 1,000, 10,000, 100,000, 1,000,000 or more, e.g., up to about 10-40 million imaging agent molecules or therapeutic molecules, e.g., from about 500 to 1,500, 2,500 to 7,500, 7,500 to 12,000, 12,000 to 50,000, 50,000 to 250,000, 250,000 to 500,000, 500,000 to 750,000 or 750,000 to 1,250,000, imaging agent molecules or therapeutic molecules. In one embodiment, the surface of the macrophage is not modified to include a nanoparticle, e.g., a quantum dot or dendimers. In one embodiment, the surface of the macrophage is not contacted with an inorganic salt in order to modify the surface to include an imaging agent or a therapeutic agent. In one embodiment, the surface of the macrophage is modified to include a prodrug.

Also provided is a method to prepare a macrophage-based imaging agent or therapeutic agent. The method includes providing a population of isolated primary mammalian macrophages and chemically modifying the cell surface of the macrophage to include an imaging agent or a therapeutic agent, or a molecule that binds to an imaging agent or a therapeutic agent. In one embodiment, the modified macrophages are introduced into a mammal. In one embodiment, the macrophages are autogeneic to the mammal. In one embodiment, the macrophages are allogeneic to the mammal.

A method for imaging in a mammal is further provided. A population of isolated mammalian macrophages, the surface of which is chemically modified with an imaging agent or a molecule that binds to an imaging agent, is introduced to a mammal. In one embodiment, the agent is detectable in vivo after exposure of the mammal to energy and optionally a composition comprising a metal. In one embodiment, X-rays or a magnetic field are applied to the mammal and images of the population in the mammal recorded. In one embodiment, the images are analyzed. In one embodiment, the modification comprises a chelator or an azide and phosphine functionalized sugar. In one embodiment, the macrophages are autogeneic to the mammal.

Further provided is a method to inhibit or treat cancer in a mammal. The method includes providing a composition comprising a population of isolated mammalian macrophages, the surface of which is chemically modified with a chemotherapeutic agent, and introducing an effective amount of a composition having the population into the mammal. In one embodiment, the macrophages are autogeneic to the mammal. In one embodiment, the macrophages are allogeneic to the mammal.

Also provided is a method to inhibit or treat cardiovascular disease, e.g., treat plaques or inhibit or prevent their formation, in a mammal. The method includes providing a composition comprising a population of isolated mammalian macrophages, which are optionally modified to inhibit or prevent expression of CD36 and/or TLR4, and optionally to enhance expression of CCR7, the surface of which is chemically modified with a decoy receptor and/or a target kill switch, and introducing an effective amount of a composition having the population into the mammal. In one embodiment, the macrophages are autogeneic to the mammal. In one embodiment, the macrophages are allogeneic to the mammal. In one embodiment, a linker is employed to modify the surface of the macrophage with the decoy receptor.

### BRIEF DESCRIPTION OF FIGURES

FIG. 1. Macrophages as cell-based delivery agents.

FIGS. 2A-B. Macrophage drug delivery and linker strategy. A) Chemotherapeutics to be attached to macrophage surfaces. Yellow boxes indicate functionalities present in drug molecules, while pink boxes represent entities that enable further chemical modification. B) Schematic of cell surface modification with cleavable linkers to separate drugs from macrophages.

FIG. 3. Chemical modification of macrophages with fluorescent labels. Schematic of biotin/streptavidin versus metabolic labeling strategies. Biotin/streptavidin uses NHS-biotin to couple to lysine residues on the cell surface, followed by biotin-streptavidin-dye conjugate association. The metabolic labeling approach is based on incorporation of azide functionalized sugars into membrane glycoproteins, followed by chemical reaction with phosphines. Macrophage labeling is both dependent on the chemical functionalization of the cells (no non-specific association with fluorophore is observed) and proceeds via both biotin/streptavidin and metabolic strategies. RAW264.7 and J774.2 and primary (bone derived) macrophages have been modified.

FIG. 4. Scratch assay of avidin-FITC labeled RAW264.7 cells compared with non-modified macrophages. Both conditions resulted in similar growth and migratory behavior. The white dashed line indicates the front of the densest cell area, however significant migration of individual cells is apparent within the ‘wound’ for both after 24 hours.

FIGS. 5A-B. A) Under-agarose migration assay of avidin-FITC labeled RAW264.7 cells compared with non-modified macrophages. Agarose droplets were generated with or without the macrophage chemoattractant Colony Stimulating Factor 1 (CSF-1). PBS=unmodified macrophages, no CSF-1. CSF-1=unmodified macrophages, with CSF-1. FITC=modified macrophages, with CSF-1. Modified cells showed about 75% of the migratory capacity compared to non-modified cells. B) Association of macrophage with cancer cells.

FIGS. 6A-B. In vitro and in vivo behavior of modified macrophages. A) RAW264.7 macrophages (green) with MCF7 breast cancer cells. B) Ex vivo biodistribution of Dylight 680-labeled RAW264.7 macrophages 24 hours following tail-vein injection, showing tumor localization and penetration.

FIGS. 7A-F. A) Rationale for use of patient-derived macrophage to treat cardiovascular disease (CVD). B) Exemplary macrophage modifications to provide autologous cells that have a reduced pro-inflammatory response. C) Surface modifications to macrophage to allow for tethering of decoy receptors. D) Macrophage having imaging agents for high contrast imaging. E) Gold nanoparticles tethered to macrophage for imaging. F) At site of unstable plaques. The modified macrophage release, e.g., surface tethered decoy receptors to disrupt pro-inflammatory signaling and optionally to enhance egress of resident macrophage. E) Removal of modified macrophage via apoptosis.

FIGS. 8A-C. A) Exemplary linkers. B) Alternative strategies for labeling. C) Exemplary molecules for conjugation.

FIGS. 9A-C. A) Migration assay of unmodified and FITC modified macrophage in the presence or absence of CSF. B-C) Images of unmodified and surface modified macrophage in the presence of CSF.

FIGS. 10A-B. A) Migration assay of unmodified and surface modified (NHS-Cy5, Phos-DY650) macrophage in the presence of CSF relative to control. B) Images of unmodified and surface modified (NHS-Cy5, Phos-DY650) macrophage in the presence of CSF.

FIGS. 11A-B. Generation of phenotypic reporters for studies of macrophage association with cancer subtype. A) Strategy for use of macrophages in re-education studies (blue and green represent two different fluorescent protein fusions). B) Differential protein abundance based on macrophage phenotype.

FIG. 12. Exemplary molecules for macrophage re-education (targets shown in parentheses).

### DETAILED DESCRIPTION

The present disclosure relates to the use of macrophage characteristics and manipulation for: (1) conversion to and redirection from the Tumor-associated macrophage (TAM) phenotype and (2) their use as delivery agents for therapeutics, e.g., cancer therapeutics. TAMs have been associated with a variety of tumor types, including breast, prostate, glioma, lymphoma, bladder, lung, cervical, and melanoma. Macrophage-based reporters may be deployed in conditions mimicking the tumor microenvironment, enabling visualization of macrophage interconversion. By using small molecules to target cellular factors associated with macrophage polarization, macrophage phenotypes can be re-directed, from immune-suppressing, tumor-promoter subtypes to immune-activating, cancer-fighter phenotypes. Taking advantage of macrophages' innate abilities to home to cancers, those macrophage can be converted into cell-based delivery agents via surface modifications.

One of the most exciting areas of cancer study and treatment today occurs at the intersection between the disease and the immune system. A promising breakthrough in the field is the development of ‘adoptive cell transfers,’ where T-cells from cancer patients are isolated, propagated, and/or re-activated to produce additional cancer fighting cells, and infused back into the body (Wang and Riviere, 2015). With this progress, the time is ripe for the development of cell-derived therapies and reporters that can detect and take advantage of inherent characteristics of cancer. Macrophages are another major immune cell type, whose phenotype depends on location and the physiological or pathological context present. Tumor associated macrophages (TAMs) have been correlated with tumor angiogenesis, invasion and metastasis, and poor patient prognoses (Mukhtar et al., 2011). TAMs generate factors that promote tumor angiogenesis, silence the immune response to tumors, contribute to the metastasis-enabling processes of microenvironment remodeling and intra- and extravasation of migratory tumor cells (Mukhtar et al., 2011). It has been shown that macrophages are not only important in initial stages of metastasis but also contribute to metastasis establishment and survival at distant sites (Qian et al., 2009). TAMs have been associated with many cancers, including breast, prostate, glioma, lymphoma, bladder, lung, cervical, and melanoma; their levels have been correlated with malignancy and poor prognoses. In some breast cancers, these macrophages may comprise up to 50% of the tumor cell mass (Zhang et al., 2013). Tumor cells produce two of the major attractants and growth factors for TAMs, colony-stimulating factor (CSF)-1 (Pixley and Stanley, 2004) and CCL2 (Qian et al., 2011). While there are many research groups interested in the development of ‘targeted’ imaging agents and therapeutics, all of these entities first require passive accumulation at the site of interest. Only then can the ‘targeting group’ directly interact with cell surface receptors or other local markers to facilitate binding or other specificity-related activities. In contrast, the present platform takes advantage of the fact that macrophages are being actively recruited to sites of disease.

Linker-conjugated fluorophores may be used to confirm continued chemotactic response and payload release under appropriate conditions, both in vitro and in vivo, using a dual-fluorophore strategy. While one fluorophore is attached to the cell via a cleavable linker, the other is stably attached. This enables the screening of linkers by determining cleavage extent via co-localization. Concurrently, macrophage-drug conjugates (also bearing fluorophores) are evaluated for cytotoxic effects on the macrophage cells. Their abilities to localize to and shrink tumors in vivo may be assessed by using the 4T1:luciferase tumor model. The respective macrophage therapeutics may be compared with non-conjugated drugs, macrophages covalently linked to small molecules (non-cleavable), and non-modified macrophages.

As described herein, chemical methods were employed to modify macrophage surfaces, e.g., via chemical conjugation or metabolic labeling, yielding agents that are able to detect primary tumors and metastases, e.g., for detection, after administering a detectable probe that binds to the surface modification, or for therapy to selectively deliver drugs. For example, macrophages were labeled with fluorophores via two different strategies, and those macrophages retained the ability to respond to chemoattractant signals produced by cancers. These reagents may be used for, for example, metastasis targeting, and macrophage-based drug delivery via incorporation of cleavable linkers and therapeutic agents. Together, these theragnostic agents (part diagnostic, part therapeutic) may detect and treat disease, not only primary tumors, but also nascent metastases.

### Exemplary Agents for Imaging

Nuclear imaging, magnetic resonance imaging (MRI), magnetic resonance spectroscopy, computed tomography (CT), ultrasound (US), bioluminescence, fluorescence imaging (optical imaging), and photoacoustic imaging are employed for imaging in vivo. The success of an imaging modality depends on a combination of various factors. Along with the issues of biocompatibility, toxicity and probe stability, another challenge associated with the use of various imaging modalities is to achieve a high contrast signal over nearby tissues. Each modality has certain advantages and/or disadvantages. For instance, Magnetic Resonance Imaging (MRI) in general has low sensitivity, relatively long imaging times, and uses large amounts of injected contrast agents. However, MRI provides high image resolution and exquisite soft tissue contrast for revealing tissue morphology and anatomical details. Certain modalities, such as Positron Emission Tomography (PET), employ radioisotopes. PET can be employed with a wide range of radiolabeled tracers with varied half-lives, and provides for high resolution, high contrast and high sensitivity and specificity.

In one embodiment, e.g., for positron emission tomography, the cells are modified with a chelator such as a macrocyclic polyamino carboxylate (e.g., DOTA and TETA), or an acyclic chelating agent (e.g., EDTA and DTPA). The chelator may bind or be specific for ⁶⁴C, ¹³N, ⁷⁶Br, ⁸⁶Y, ⁶⁸Ga, ⁸⁹Zr, ^{94m}Tc, ¹²⁴I, 11C, ¹⁸F or ¹⁵O. Alternatively, ⁶⁴C, ¹³N, ⁷⁶Br, ⁸⁶Y, ⁶⁸Ga, ⁸⁹Zr, ^{94m}Tc, ¹²⁴I, 11C, ¹⁸F or ¹⁵O, or other molecules, may be directly attached to the cell surface via compatible linker chemistries. For MRI, the cells may be modified to include chelators of lanthanides, such as Gd, e.g., a hydroxypyridonate, DTPA, HOPO, TR322, TR332, TPPN, HP-DO3A, DO3A-butrol, DTPA-BMA, DTPA-BMEA, BOPTA, EOB-DPTA, MS-325 or DOTA.

### Exemplary Therapeutic Agents

Exemplary therapeutic agents useful in the modifications and method include but are not limited to nucleic acid, e.g., dsDNA, plasmid DNA, siRNA, shRNA, or microRNA, or drugs including everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafamib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox,gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, or a mixture thereof.

### Linkers

For example, one type of functionalization is to immobilize molecules that include an imaging agent or a therapeutic agent, or molecules that bind to an imaging agent or a therapeutic agent, onto the surface of macrophage, optionally using a linker. Chemical cross-linking (conjugation), e.g., using linkers, may be used for covalently immobilizing molecules, e.g., chelators, to the macrophage surface. The molecule that is crosslinked to the surface may aid in detection of the macrophages, e.g., indirectly if another molecule is needed, e.g., a metal that complexes with a chelator that is covalently linked to the cell surface. With respect to cross-linking of a linker to proteins on the cell surface, four protein chemical targets may be employed: primary amines (—NH2): this group exists at the N-terminus of each polypeptide chain and in the side chain of lysine (Lys, K) residues; carboxyls (—COOH): this group exists at the C-terminus of each polypeptide chain and in the side chains of aspartic acid (Asp, D) and glutamic acid (Glu, E); sulfhydryls (—SH): this group exists in the side chain of cysteine (Cys, C). Often, as part of a protein's secondary or tertiary structure, cysteines are joined together between their side chains via disulfide bonds (—S—S—); and carbonyls (—CHO): these aldehyde groups can be created by oxidizing carbohydrate groups in glycoproteins. For each of these protein functional group targets, there exists one to several types of reactive groups that may be used as the basis for crosslinking and for modification reagents. All of these groups are available for use with the particles described herein.

Thus, in addition to the functionalization described herein molecules that increase molecular mass, increase solubility for storage, or create a new functional group that can be targeted in a subsequent reaction step. For example, cells may be PEGylated by chemically attaching single- or branched-chain polyethylene glycol (PEG) groups to proteins, which provides for labeling, enhanced water-solubility and/or addition of inert molecular mass to proteins. In another example, cells may be modified with block sulfhydryls. Proteins can include sulfhydryls (e.g., the side chain of cysteine) and certain reagents are capable of reacting permanently or reversibly with sulfhydryl groups (e.g., methylmethanethiosulfonate, MMTS, and N-ethylmaleimide, NEM, respectively). Other modifications include the conversion of amines to a sulfhydryl-containing group to the primary amine. N-succinimidyl S-acetylthioacetate, SATA, and related reagents contain an amine-reactive group and a protected sulfhydryl group. By reacting the compound and a protein, the side chain of lysine residues can be modified to contain a sulfhydryl group for targeting with sulfhydryl-specific crosslinkers or immobilization chemistries. The effect is also to extend the length of the side chain by several nanometers.

In one example, agents may be attached onto the surface using the chemical cross-linker dithiobis(succinimidyl propionate) (DSP, Product No. 22585, Pierce Biotechnology). DSP is a homobifunctional, amine-reactive cross-linker. The disulfide linkage in DSP chemisorbs rapidly to surfaces, while the active NHS groups on either end of DSP are reactive toward primary amine groups in proteins.

In one embodiment, cells are modified with a carboxyl moiety on the surface to act as a universal capture molecule. For instance, N-(trimethoxysilylpropyl)ethylene diamine triacetic acid addition, either together or after TEOS is employed. Alternatively, 3-aminopropyltrimethoxysilane modification followed by carboxyl group introduction with the linker elongation, (see, e.g., Schiestel et al. (2004). The attachment of proteins may be accomplished using, e.g., 1-Ethyl-3-[3-dimethylaminopropyl]carbodiimide hydrochloride (EDC or EDAC) with N-hydroxysuccinimide (NETS) or its sulfo-derivative (sulfo-NHS), a zero-length crosslinking agent used to couple carboxyl groups to primary amines.

Exemplary acid labile linkers, e.g., useful to deliver chemotherapeutic agents, include but are not limited to an azidomethyl-methyl maleic anhydride or carboxamide or hydrazone or Schiff base or thiomaleic acid based linker.

Exemplary MMP peptide substrates, e.g., useful to deliver chemotherapeutic agents, include but are not limited to Pro-X-X-Hy-(Ser/Thr) for MMP-9 and [PXXX_{Hy}], e.g., [I/LXXX_{Hy}], [X_{Hy}SXL] or [HXXX_{Hy}], wherein X_{Hy} is a hydrophobic residue, K*PAGLLGC, K*AGLLC, or for MMP-2 K*GLC, and GGGVPLSLYSGGGG or DGGDGGDGGDGPLGLAGrrrrrrrrrC (where lowercase letters represent D-amino acids) for MMP-2.

### Exemplary Methods

The ability of macrophage-based imaging and therapeutic delivery agents to home to cancers and function in vivo provides for their utility. While cell culture models are tunable and easily generated and used, they are not able to fully represent organismal physiology. By using transplant models, the same cell line may be used for both in vitro and in vivo studies. For example, the 4T1 mouse mammary carcinoma cell line is such a line, it is highly tumorigenic, invasive, and can spontaneously metastasize from the primary tumor in the mammary gland to multiple distant sites (Bailey-Downs et al., 2014), enabling the assessment of macrophage accumulation and therapeutic effect in both primary tumors and metastases. The cell line can be implanted in immunocompetent Balb/c mice, and because it is isogenic with the RAW264.7 and J774.2 macrophage cell types, the same model cell lines can be used in vitro and in vivo. To determine the location of metastases in this model, 4T1-luciferase cells, which are luminescent upon dosage with luciferin, may be used in mammary fat pad implantations. Both luminescence and fluorescence signals allow for determination of the co-localization of the macrophages with the metastatic sites. Imaging may be conducted using an IVIS optical system with computed tomography (CT). Agent accumulation in organs and in tumor/metastases may be assessed by imaging over a variety of time-points and performing ex vivo bio-distribution following the final time-point.

In one embodiment, primary macrophages, e.g., a patient's own macrophage (or otherwise compatible) are isolated, cultured, and modified. In one embodiment, macrophage-based imaging agents are prepared that may be used with diagnostic imaging modalities, e.g., positron emission tomography (PET) and magnetic resonance imaging (MRI). For example, the surface of macrophages as modified using a chelator for Cu-64 (for PET), including DOTA or NOTA, or may be modified using for gadolinium (for MRI), or other lanthanides, modified to include hydroxypyridonate complexes, e.g., AAZTA and DPDP.

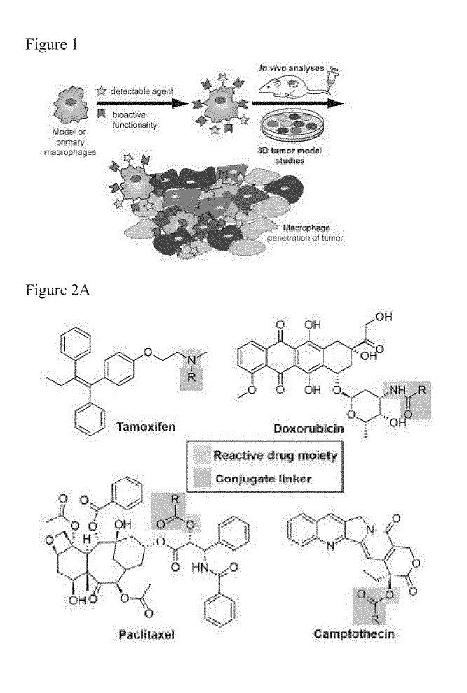

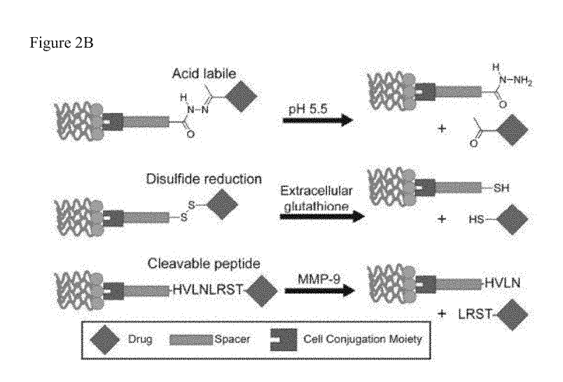

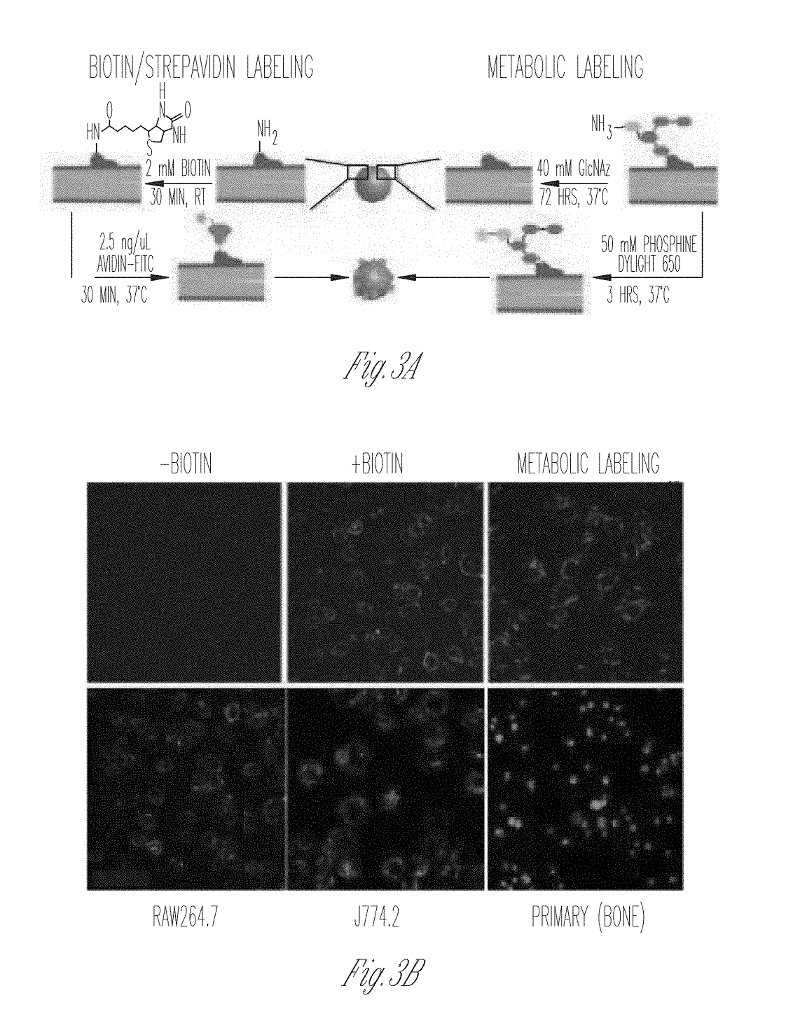

In one embodiment, macrophages are modified into therapeutic delivery agents. Broadly cytotoxic cancer drugs may be appended to cell surfaces via a cleavable linker/spacer that releases the drugs within the tumor microenvironment. These conjugates home to and ablate cancer cells. Two exemplary conventional chemotherapeutics are used to demonstrate the effectiveness of the cell-based delivery platform: tamoxifen (Xanthopoulos et al., 2005) and doxorubicin (ElBayoumi et al., 2009), both of which have shown efficacy in the 4T1 tumor model. Both drugs have been conjugated to other macromolecular entities (e.g., nanoparticles or proteins), with retention of activity (FIG. 2A) (Dreaden et al., 2009; Yu et al., 2014). Unlike imaging agents, therapeutic compounds have intracellular targets and so in one embodiment cleavable linkers are employed, which under conditions present in the tumor microenvironment, facilitate release of the molecule from the macrophage surface. Two such characteristics include the acidic nature of tumor microenvironments (pH 4.5-6.9; bloodstream pH is approximately 7.4), and the presence of increased levels of matrix metalloproteinases (MMPs). Acid labile linkers include hydrazones but are not limited to (Yang et al., 2010), Schiff bases (Yu et al., 2014) and thiomaleamic acids (Castaneda et al., 2013), which are all responsive in the range of physiologically acidic pHs, and have been used as labile linkages in a variety of drug delivery systems, including with nanoparticles, micelles, and cell penetrating moieties for tumor microenvironment-specific release of bioactive molecules. Peptide-based linkers containing sequences susceptible to cleavage by MMP-2 and MMP-9, found in tumor environments may also be used as a secondary strategy (Shi et al., 2012). These linker types are compatible with methods for macrophage cell surface modification (FIG. 2B).

The invention will be described by the following non-limiting examples.

### EXAMPLE I

A. Cell Surface Functionalization of Macrophages with Detectable Entities.

The surfaces of macrophages were chemically modified with trackable, fluorescent moieties. The cells were functionalized using two different strategies (FIG. 3). One method used N-hydroxysuccinimide-functionalized biotin (sulfo-NHS-LC-biotin) to modify cell-surface proteins via reaction with accessible lysine R-group amines, followed by reaction with streptavidin-linked fluorophores. While macrophages are phagocytic cells, no non-specific interactions between avidin-fluorescein isothiocyanate (FITC, fluorophore) were observed; inclusion of the dye moiety only occurred following biotin functionalization (FIG. 3). In another embodiment, metabolic incorporation of cellular glycans with azide-functionalized sugars for subsequent reaction with phosphine-dye conjugates or other ‘clickable’ reagents, e.g., alkyne-based reagents, may be used. In another embodiment N-hydroxysuccinimidyl esters may be used for modification of amine functional groups. In another embodiment maleimides may be used for modification of cysteine functional groups. Labeling occurred only in the presence of both conjugation moieties. On average, each cell, including RAW264.7 (Abelson murine leukemia), J774.2 (monocyte), and primary bone-derived macrophages (isolated from Balb/c mice) (FIG. 3), was modified with hundreds of millions of fluorophores.

### B. Evaluation of Macrophage Behavior Following Modification

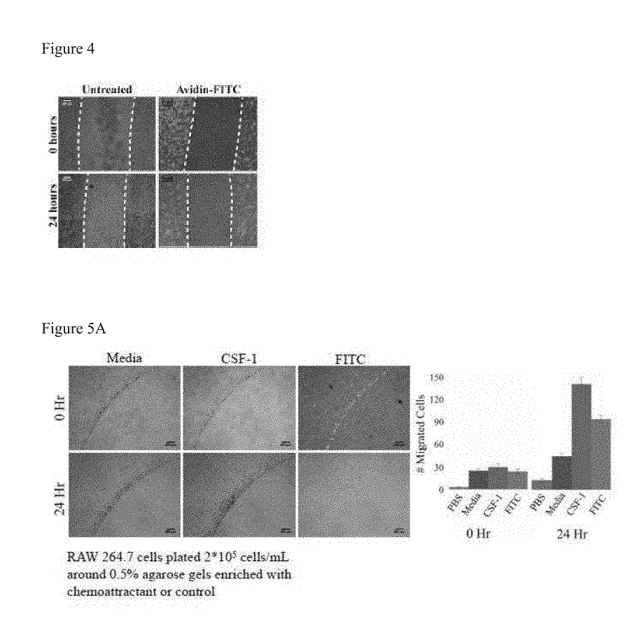

Wound-healing/scratch assays, where cells are grown followed by removal within an area (scratch), showed similar levels of growth and migration between cell conjugates and untreated cells (FIG. 4). Thus, surface modifications do not appear to alter motility of the macrophages.

Two of the major macrophage homing signals secreted by cancers are colony stimulating factor 1 (CSF-1) and CCL2. Under agarose migration assays were used to assess the chemotactic responses of surface modified versus non-treated macrophages. Agarose droplets containing CSF-1 or PBS (for control) were positioned in cell culture plates, and macrophage response to stimulus was determined by tracking cell movement toward and into the agarose (FIG. 5). Over 24 hours, the majority of the modified and un-modified cells was found to migrate to the agarose front, with more of the native cells trafficking into the agarose. Minimal changes were observed for any cells exposed to agarose-PBS only. The labeled cells showed approximately 75% of the native response.

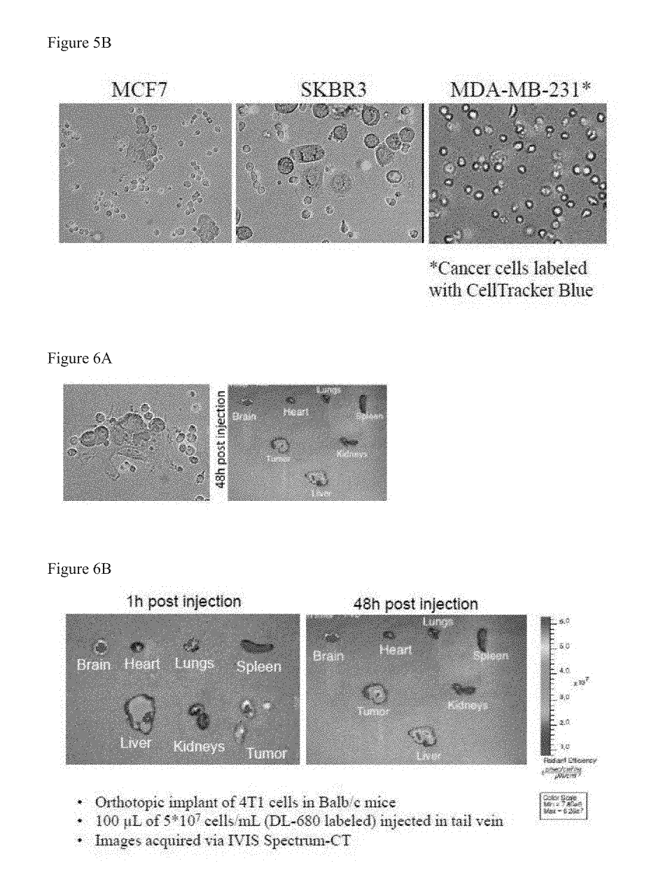

In two-dimensional co-culture experiments with cancer cells representing different breast cancer subtypes, modified macrophages were shown to be attracted to, make contact with, and manipulate the cells (FIG. 6). The 4T1 mouse mammary carcinoma cell line is highly tumorigenic, invasive, and can spontaneously metastasize from the primary tumor in the mammary gland to multiple distant sites (Bailey-Downs et al., 2014), enabling the assessment of macrophage accumulation and therapeutic effect in both primary tumors and metastases. It can be implanted in immunocompetent Balb/c mice, and is isogenic with the RAW264.7 macrophage cell type. RAW264.7 macrophages labeled with near infrared dye (facilitating tissue penetration) were able to localize to 4T1 orthotopic tumors. Therefore, macrophage-based imaging and therapeutic delivery agents home to and interact with cancers.

In summary, macrophages were converted into cell-based imaging agents and/or drug-delivery agents, e.g., that home to tumor sites. The diagnostic entities are sensitive and the homing of the macrophages ameliorates widespread toxicity. Chemically-modified macrophages accumulate at both primary tumor and metastatic sites, and can be functionalized with drugs conjugated via cleavable linkers that facilitate release upon encountering conditions present in the tumor microenvironment.

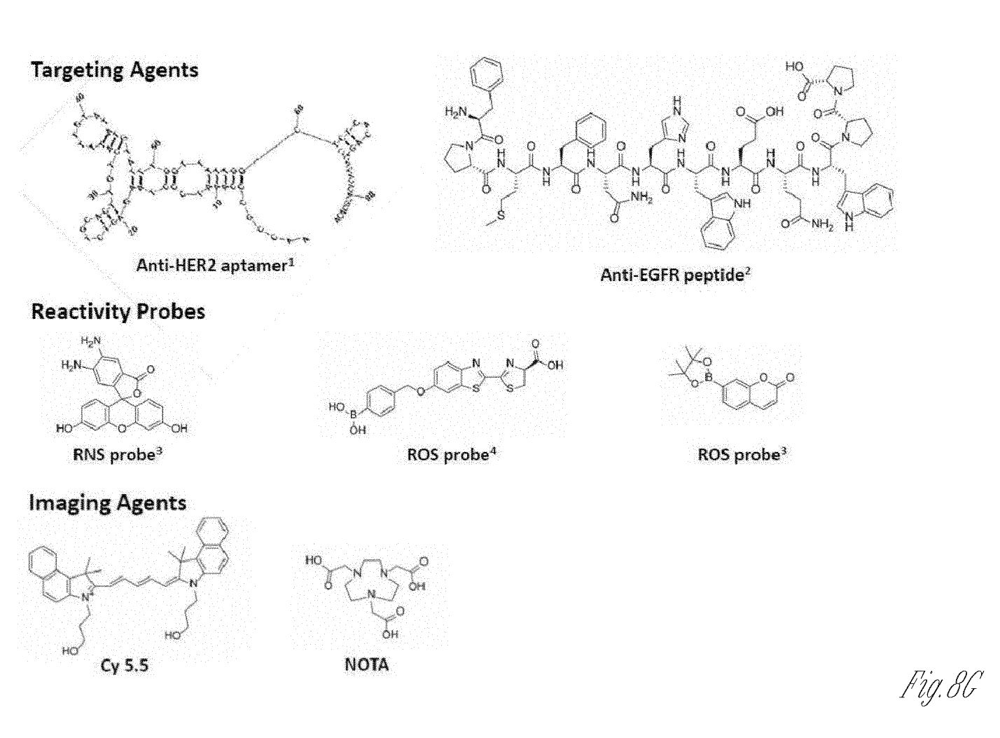

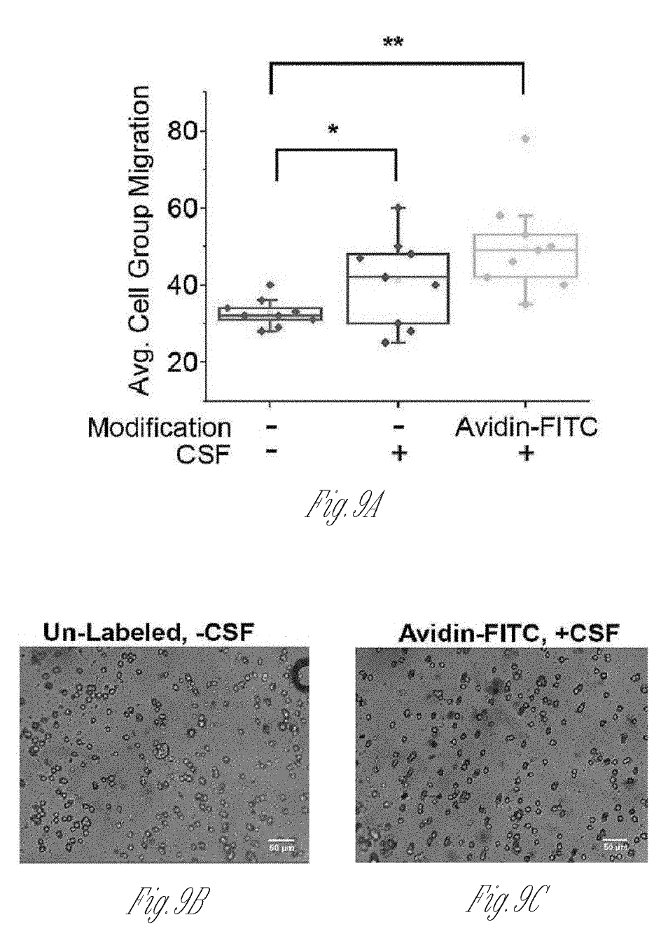

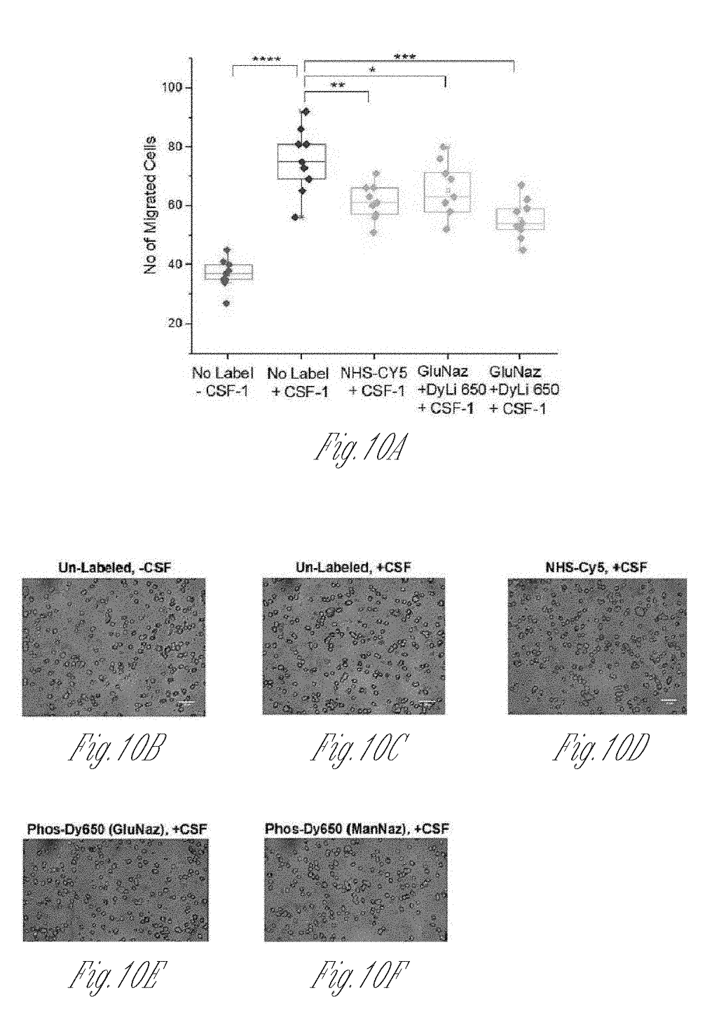

Modified macrophage behave similarly to unmodified cells, including migration capacity, response to chemoattractants (see FIGS. 9-10), and interactions with cancer cells.

### EXAMPLE II

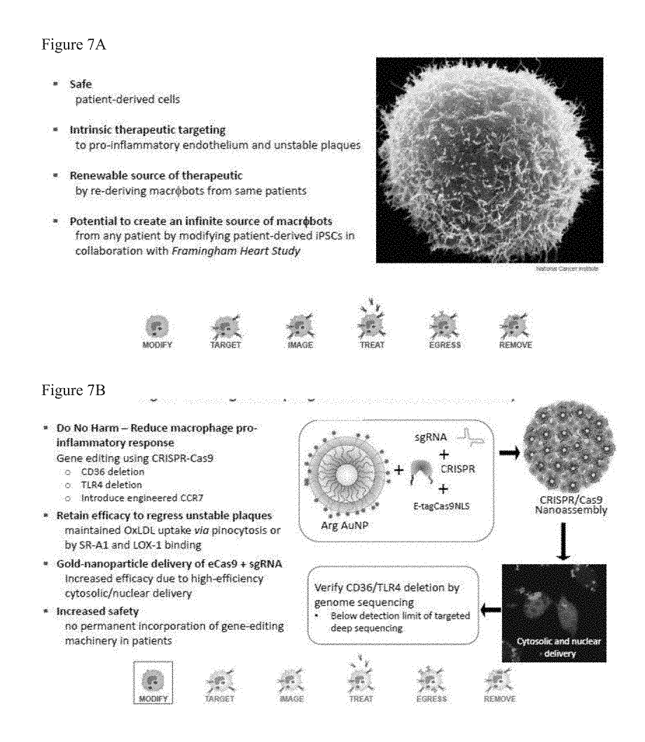

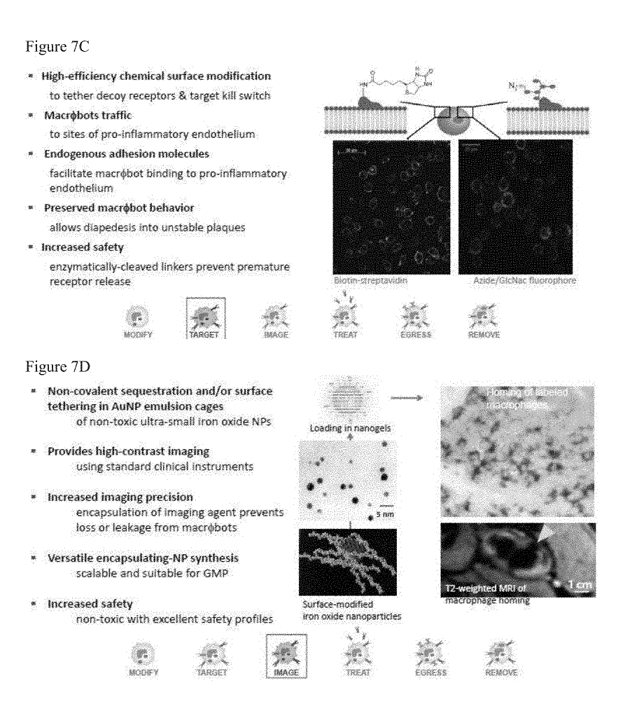

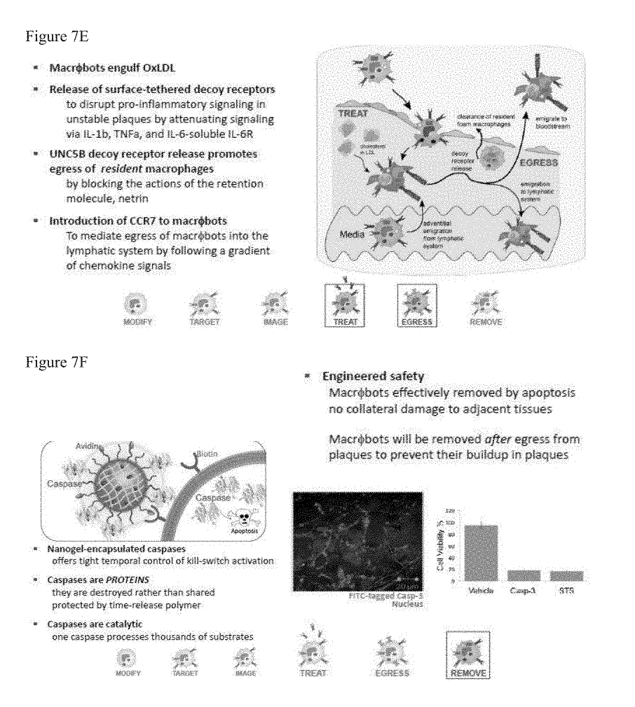

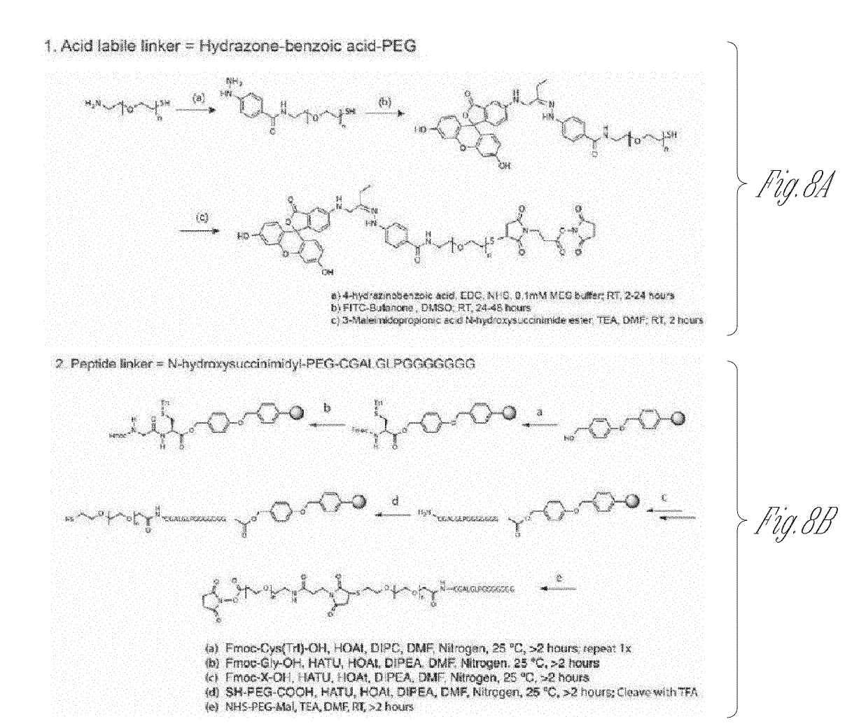

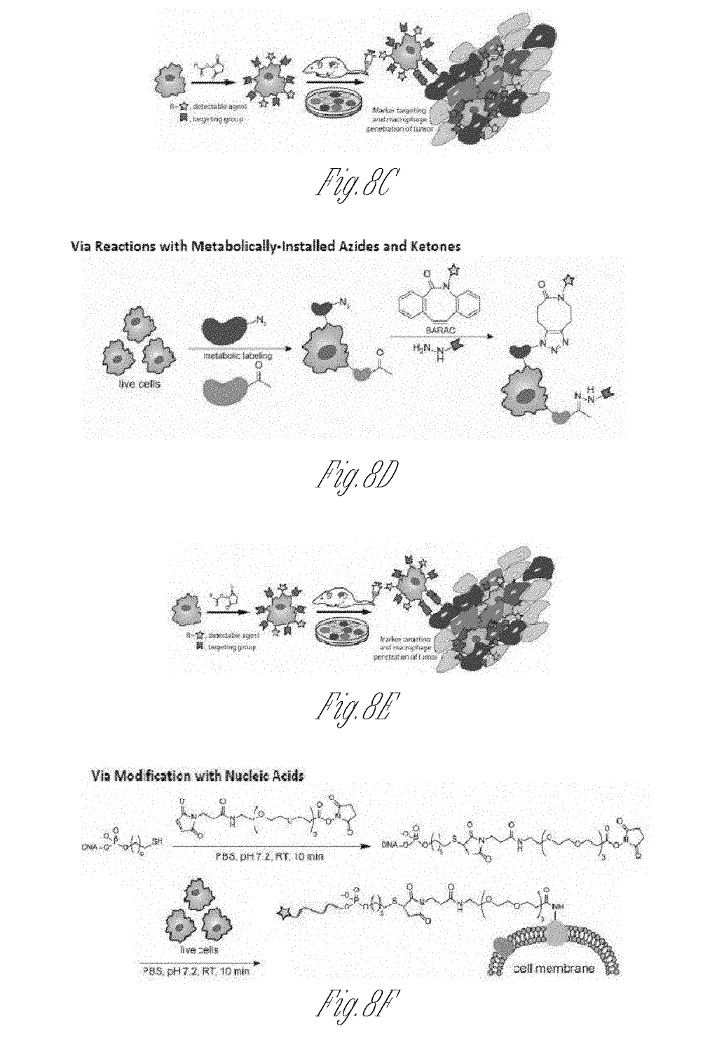

In one embodiment, macrophage can be modified to treat cardiovascular disease, e.g., heart attacks and strokes, by targeting unstable plaque (FIG. 7A. For instance, macrophage may be modified to reduce the pro-inflammatory response but retain the ability to regress unstable plaques (FIG. 7B). FIG. 7C shows chemical surface modification of macrophage and FIG. 7D shows macrophage having imaging agents that may be sequestered in the macrophage or linked to the cell surface of macrophage. FIG. 7E illustrates the use of decoy receptors to promote egress of resident macrophage, and FIG. 7F shows an embodiment where the modified macrophages can be eliminated from the host.

The imaging agent surface modified macrophage target by adhering and diapedesis through pro-inflammatory endothelial cells and targeting ECM embedded oxLDL. The modification allows for imaging and tracking of individual cells. The release of decoy receptors may be observed. Macrophage egress may occur via migration towards a chemokine gradient and caspase activation may allow for removal of the cells. The surface modified macrophage may be employed to remove plaque, e.g., carotid plaque or coronary plaque.

### EXAMPLE III

For direct NHS labeling, the cells may be directly functionalized with NHS fluorophore (or small molecule) conjugates, where the amines at the cell surface are coupled to the NHS esters. An example protocol for modification of cells with Sulfo-NHS-Cyanine5 follows. Cell monolayer was treated with 0.25% trypsin for detachment, and 4×10⁶ cells were centrifuged at 1500 rpm for 5 minutes. The pellet was rinsed twice with 1 mL PBS, centrifuging and removing supernatant for each wash, resuspended in 400 μL of 100 μM Sulfo-NHS-Cyanine5, and incubated for 1 hour at 37° C./5% CO₂. Cells were centrifuged at 1500 rpm for 5 minutes, and the pellet was rinsed twice with 2 mL of 100 mM glycine, and once with 1 mL of PBS before use.

### EXAMPLE IV

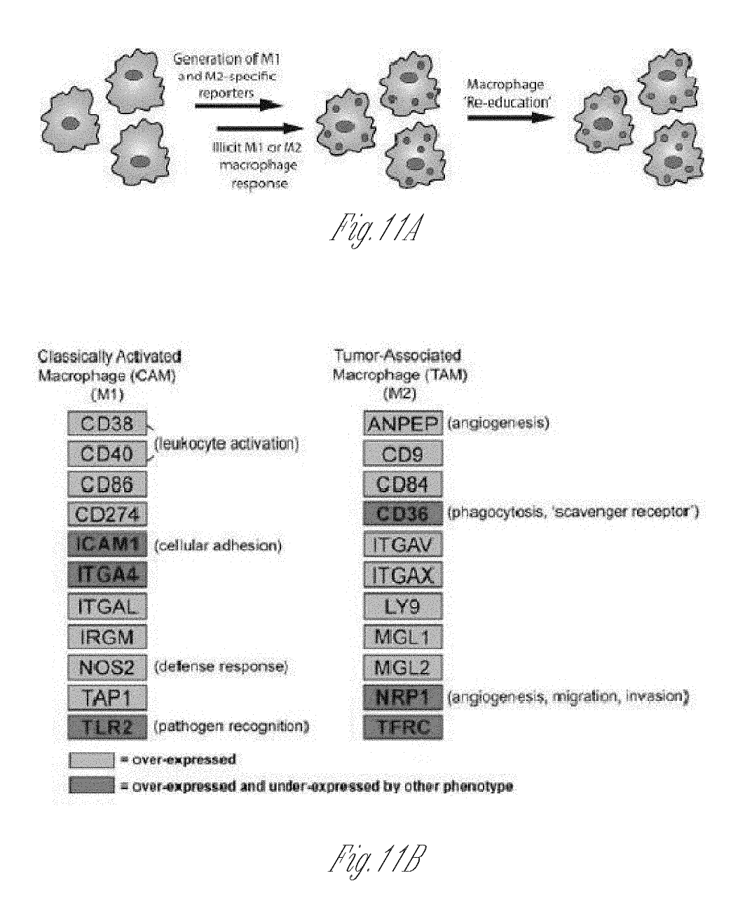

A report system may distinguish between macrophage states, yielding spatial and temporal resolution (FIG. 11A). It is known that conditioned media from malignant epithelial cells can polarize macrophages to an M2-like phenotype (pro-tumoral), and macrophages can also be “re-educated” to reverse their tumor promoting activity and produce an anti-tumor M1 phenotype. However, currently the only mechanisms available for macrophage phenotype identification in re-education studies involve the analysis of culture media, visualization of macrophage morphology, and isolation of cells for protein/mRNA/immunofluorescence analysis. None of these means are amenable to real-time analysis of conversion in complex disease model environments, re-enacting the roles of macrophages in invasion and metastasis, and determining the conditions for reprogramming TAMs into tumor fighting entities. Fluorescent protein reporters were prepared based on proteins differentially expressed between the two major phenotypes (FIG. 11B). Two different types of fluorescence are generated, which will depend on and change with the macrophage status.

Markers to be used for phenotypic reporters are ICAM1 (Intercellular Adhesion Molecule 1) and TLR2 (Toll-Like Receptor 2), overexpressed by M1 macrophages, and CD36 (Cluster of Differentiation 36) and NRP1 (Neuropilin 1), overexpressed by M2 macrophages. All of these proteins have well-characterized promoter elements, and readily lend themselves to the generation of fluorescent protein fusions. The M1-associated genes are cloned into lentiviral plasmids with green fluorescent protein (GFP) tags, while M2-assocaited genes are cloned into lentiviral plasmids with mCherry (a variant of red fluorescent protein) tags. The gene inserts for all four genes of interest (obtained from ThermoScientificBio and Addgene) have already been amplified out of the murine-derived plasmids and ligated into intermediary (‘suicide’) vectors (CloneJET, Thermo Scientific). Murine RAW264.7 (Abelson murine leukemia) and J774.2 (monocyte) immortalized macrophage cell lines and isolated primary macrophages from bone and peritoneum may be used.

### EXAMPLE V

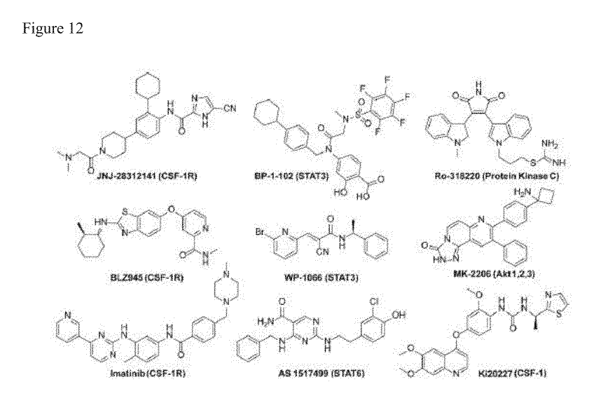

To identify bioactive molecules that re-direct macrophage phenotypes, small molecule antagonists are used to target factors associated with macrophage polarization and tumor attraction. CSF-1 (colony-stimulating factor-1) is one of the major chemoattractants for macrophages and is produced by breast tumor cells. It has also been shown to mediate the differentiation of monocytes into M2 macrophages in vitro. When deleted, macrophage maturation and tumor infiltration is inhibited, and angiogenesis and malignant progression are delayed. Small molecule inhibitors of CSF-1 have been investigated as cancer therapies, but their direct effects on macrophages have not been studied. STAT3 is a downstream target of IL-6, and is part of the positive feedback loop that underlies the epigenetic switch linking inflammation to cancer. It is found in both tumors and tumor-infiltrating macrophages and is required for the transformed state in diverse series of cell lines and tumor growth in xenografts. M2 macrophages have been shown to exhibit M1 behavior in the presence of STAT3 inhibition. Other macrophage targets of interest include: mitogen-and stress-activated protein kinase (MSK), which controls the phosphorylation of an inducer of M2 phenotype (Mg); protein kinase B (AKT), which can inhibit IL-4 inducible phosphorylation, leading to reduced levels of M2 activation-specific cascades; and STAT6, which can mediate programmed death ligand-2 (PD-L2) and targets IL-4 in alternately activated (M2-type) macrophages. To probe the conditions under which re-education can occur, non-polarized and M2-polarized macrophages are treated with a small bioactive molecules that are known to target factors involved with macrophage polarization and tumor attraction, e.g., inhibitors of CSF-1, CSF-1R, STAT3, MS, AKT, and STAT6 (see FIG. 12).

### EXAMPLE VI

Breast cancers are able to produce two of the major attractants and growth factors for TAMs, colony-stimulating factor (CSF)-1 and CCL2. Furthermore, it has been shown that macrophages are not only key players in the initial stages of metastasis (including EMT and intravasation), but are also present in and contribute to metastasis establishment and survival at sites distant from the primary tumor. Active recruitment of macrophages to tumor sites may result in greater selectivity and fewer off-target effects for patients. Macrophage surfaces are functionalized with molecules, e.g., detectable fluorescent molecules for cellular and in vivo imaging. For example, macrophages were modified with fluorophores using two different strategies. Modular and general approaches are desirable. In addition to macrophage, other cell-based imaging agents include macrophage precursor monocytes, red blood cells, T-cells, and other cell types. One method uses N-hydroxysuccinimide-functionalized biotin to modify cell-surface proteins via reaction with lysine R-group amines, followed by reaction with streptavidin-linked fluorophores. This results in a 5 kDa protein on the cell surface. Other methods employ metabolic labeling of cellular glycans with azide-functionalized sugars for subsequent reaction with phosphine-dye conjugates.

Macrophages are also converted into therapeutic delivery agents, by modifying their surfaces with cancer drugs. This strategy applies to molecules that are able to successfully redirect macrophage phenotype and result in anti-cancer effects. Conventional chemotherapeutics to be used here may include doxorubicin, taxol, camptothecin, and tamoxifen. Each of these drugs has previously been conjugated to other macromolecular entities (e.g., nanoparticles or proteins), with retention of activity, and permissive sites for further chemical modification are well understood.

Unlike imaging agents, therapeutic compounds need to reach their target in order to display an effect. The use of extended spacers between the bioactive molecules and the delivery macrophages, and/or the use of cleavable linkers, which under conditions present in the tumor microenvironment, may facilitate release of the molecule from the macrophage surface. For extended spacers, polyethylene glycol (PEG), a polymer that can be obtained in a variety of lengths and chemical entities at the termini, enables the use of different reactions for functionalization. It is water soluble, has low toxicity, and is used in a variety of biological applications, including the slowing of clearance from and masking immune recognition in the bloodstream. In the use of cleavable linkers, we will take advantage of the acidic nature of tumor microenvironments (pH 4.5-6.9; bloodstream pH is approximately 7.4), and presence of increased levels of matrix metalloproteinases and glutathiones as well. Acid labile linkers include hydrazones, Schiff bases, and thiomaleamic acids, which are all responsive in the range of physiologically acidic pHs, and have been used as labile linkages in a variety of drug-delivery systems, including with nanoparticles, micelles, and cell penetrating moieties for tumor microenvironment-specific release of bioactive molecules. Disulfide linkers are cleaved by glutathione (GSH). Peptide-based linkers containing sequences susceptible to cleavage by matrix metalloproteinases (2 and 9) found in tumor environments may also be used. All linker types may be amenable to use with strategies for macrophage cell surface modification. Following chemical modification of the drug molecules to incorporate linker-spacer entities, they are conjugated to the macrophage surfaces.

### REFERENCES

Mukhtar et al., Expert Rev. Mol. Diagn., 11:91 (2011).

Pixley and Stanley, Trends Cell Biol., 14:628 (2004).

Qian et al., Nature, 475:222 (2011).

Wang and Riviere, Cancer Gene Ther., 22:85 (2015)

Qian et al., PLoS ONE, 4:e6562 (2009).

Zhang et al., PLoS ONE, 8:e76147 (2013).

Bailey-Downs et al., PloS ONE, 9:e98624 (2014).

Xanthopoulos et al., J. Biomed. Biotechnol., 1:10 (2005).

ElBayoumi et al., Clin. Cancer Res., 15:1973(2009).

Dreaden et al., Bioconj. Chem., 20:2247 (2009).

Yu et al., Biomacromolecules, 15:524 (2014).

Yang et al., Bioconj. Chem., 21:496 (2010).

Castaneda et al. Chem. Commun., 49:8187 (2013).

Shi et al., Int. J. Nanomedicine, 7:1613 (2012).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A population of isolated mammalian macrophages, the cell surface of which is chemically modified to comprise a molecule that comprises an imaging agent or a therapeutic agent or a molecule that binds to an imaging agent or a therapeutic agent, wherein the cell surface modified macrophages are capable of migration and/or responding to one or more chemoattractants.

2. The population of claim 1 wherein the mammalian macrophages comprise human macrophages or immortalized macrophages.

3. The population of claim 1 wherein the imaging agent is for position emission tomography (PET).

4. The population of claim 1 wherein the imaging agent is for magnetic resonance imaging (MRI).

5. The population of claim 1 wherein the chemical modification comprises a linker.

6. The population of claim 5 wherein the linker is a cleavable linker, an acid-labile linker or comprises a disulfide bond.

7. The population of claim 5 wherein the linker comprises a hydrazone, Schiff base, PEG, N-hydroxysuccinamide, a peptide substrate, thiomaleamic acid, or any combination thereof.

8. The population of claim 7 wherein the linker comprises a peptide substrate for a matrix metalloproteinase (MMP).

9. The population of claim 8 wherein the peptide substrate is a MMP-2 or MMP-9 substrate.

10. The population of claim 1 wherein the surface modification comprises azide-functionalized sugars or ketone-modified molecules.

11. The population of claim 1 wherein the imaging agent comprises a radiolabel.

12. The population of claim 1 wherein the molecule that binds comprises a chelator.

13. The population of claim 1 wherein the therapeutic agent comprises a chemotherapeutic.

14. A method for imaging, comprising:
providing a population of isolated mammalian macrophages, the surface of which is chemically modified with an imaging agent or a molecule that binds to an imaging agent;
introducing a composition having the population to a mammal and optionally a composition comprising a metal or radioisotope;
applying x-rays or a magnetic field to the mammal and recording images of the population in the mammal; and
analyzing the images.

15. The method of claim 14 wherein the chemical modification comprises covalent linkage of a chelator to the surface.

16. A method to inhibit or treat cancer in a mammal, comprising:
providing a composition comprising a population of isolated mammalian macrophages, the surface of which is chemically modified with a chemotherapeutic agent; and
introducing an effective amount of the composition to the mammal.

17. The method of claim 16 wherein the modification comprises a linker.

18. The method of claim 17 wherein the linker is a cleavable linker, an acid-labile linker or comprises a disulfide bond.

19. The method of claim 17 wherein the linker comprises a peptide substrate for a protease, hydrazone, Schiff base, PEG, N-hydroxysuccinamide, thiomaleamic acid, or any combination thereof.

20. The method of claim 16 wherein the macrophage are human macrophage.
